SEQUENCE LISTING (22 sequences)

SEQ ID NO: 1 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 2 - DNA, 23 bp, Artificial Sequence
  synthetic oligonucleotide
cgaatgtaacagacacggtctca

SEQ ID NO: 3 - RNA, 17 nt, Artificial Sequence
  synthetic oligonucleotide
accgugucuguuacauu

SEQ ID NO: 4 - RNA, 10 nt, Artificial Sequence
  synthetic oligonucleotide
cgugucuguu

SEQ ID NO: 5 - DNA, 20 bp, Artificial Sequence
  synthetic oligonucleotide
acgagtaacgggacaaatgc

SEQ ID NO: 6 - DNA, 20 bp, Artificial Sequence
  synthetic oligonucleotide
tccctaatctatccgcctga

SEQ ID NO: 7 - DNA, 24 bp, Artificial Sequence
  synthetic oligonucleotide
aacgagctgtatgtcgtgagaatc

SEQ ID NO: 8 - DNA, 27 bp, Artificial Sequence
  synthetic oligonucleotide
atggatcatcaagctctaagaaagaac

SEQ ID NO: 9 - DNA, 20 bp, Artificial Sequence
  synthetic oligonucleotide
ataggcttgaagcagtgcat

SEQ ID NO: 10 - RNA, 11 nt, Artificial Sequence
  synthetic oligonucleotide
cugcuucaagc

SEQ ID NO: 11 - DNA, 20 bp, Artificial Sequence
  synthetic oligonucleotide
tcgtcattccattacctacc

SEQ ID NO: 12 - DNA, 24 bp, Artificial Sequence
  synthetic oligonucleotide
tactgatcgataatgccagacgaa

SEQ ID NO: 13 - DNA, 19 bp, Artificial Sequence
  synthetic oligonucleotide
cgatcaggaaatcaaccag

SEQ ID NO: 14 - RNA, 15 nt, Artificial Sequence
  synthetic oligonucleotide
gguugauuuccugau

SEQ ID NO: 15 - DNA, 21 bp, Artificial Sequence
  synthetic oligonucleotide
tccaagcagtgagtgtgagaa

SEQ ID NO: 16 - DNA, 19 bp, Artificial Sequence
  synthetic oligonucleotide
tgacagcgtgaaatcagga

SEQ ID NO: 17 - DNA, 21 bp, Artificial Sequence
  synthetic oligonucleotide
ccatcacagctcaugcttcgc

SEQ ID NO: 18 - RNA, 11 nt, Artificial Sequence
  synthetic oligonucleotide
gaagcaugagc

SEQ ID NO: 19 - protein, 16 aa, Artificial Sequence
  synthetic polypeptide
GIDEAGRGPAIGPLVV

SEQ ID NO: 20 - protein, 12 aa, Artificial Sequence
  synthetic polypeptide
LRNIGVKDSKQL

SEQ ID NO: 21 - protein, 19 aa, Artificial Sequence
  synthetic polypeptide
HKADAKYPVVSAASILAKV

SEQ ID NO: 22 - protein, 16 aa, Artificial Sequence
  synthetic polypeptide
KLKKQYGDFGSGYPSD

(19) United States
(12) Patent Application Publication
(10) Pub. No.: US 2012/0052502 A1
(43) Pub. Date: Mar. 1, 2012

(54) REAL-TIME PCR DETECTION USING STABILIZED PROBES

(75) Inventor: Jun LI, Baltimore, MD (US)

(73) Assignee: SAMSUNG TECHWIN CO., LTD., Changwon-city (KR)

(21) Appl. No.: 13/160,821

(22) Filed: Jun. 15, 2011

Related U.S. Application Data
(60) Provisional application No. 61/378,166, filed on Aug. 30, 2010.

(51) Int. Cl. C12Q 1/68; C07H 21/00

(52) U.S. Cl. 435/6.12; 536/23.1; 536/24.3

(57) ABSTRACT

A probe protector oligonucleotide is described for the improved CATACLEAVE™ probe detection of nucleic acid sequences in a test sample. The probe protector oligonucleotide is designed to be substantially complementary to the FRET pair labeled CATACLEAVE™ oligonucleotide probe. Base pairing between the probe protector oligonucleotide and the CATACLEAVE™ oligonucleotide probe coupled with chemical modification of probe sequences prevents endonucleolytic degradation of RNA sequences in the oligonucleotide probe as well as the formation of spurious RNA:DNA hybrids during PCR cycling that can lead to non-specific cleavage of the oligonucleotide probe prior to target nucleic acid sequence detection. The improved detection method is fast, accurate and suitable for high throughput applications. Convenient, user-friendly and reliable kits are also described for the high throughput detection of target nucleic acid sequences.

### CROSS-REFERENCE TO RELATED APPLICATIONS

This application claims priority from U.S. Provisional Patent Application No. 61/378,166, filed on Aug. 30, 2010, the contents of which are hereby incorporated by reference in their entirety.

### FIELD

A stabilized oligonucleotide probe composition is disclosed for real-time PCR amplification of target nucleic acid sequences.

### DETAILED DESCRIPTION

The ability to measure the kinetics of a PCR reaction in combination with PCR techniques promises to facilitate the accurate and precise measurement of target nucleic acid sequences in a sample with the requisite level of sensitivity. In particular, fluorescent dual-labeled hybridization probe technologies, such as the “CATACLEAVE™ endonuclease assay (described in detail in U.S. Pat. No. 5,763,181; see FIGS. 1 and 2), permit the detection of PCR amplification in real-time. Detection of target sequences is achieved by including a CATACLEAVE™ probe in the amplification reaction together with RNase H. The CATACLEAVE™ probe, which is complementary to a target sequence within the PCR amplification product, has a chimeric structure comprising an RNA sequence and a DNA sequence, and is flanked at its 5′ and 3′ ends by a detectable marker, for example FRET pair labeled DNA sequences. The proximity of the FRET pair's fluorescent label to the quencher precludes fluorescence of the intact probe. However, annealing of the probe to the PCR product generates an RNA:DNA duplex that can be cleaved by RNase H present in the amplification buffer. Cleavage of the duplexed RNA sequences results in the separation of the fluorescent label from the quencher and a subsequent emission of fluorescence.

### SUMMARY

A probe protector oligonucleotide is described for the improved CATACLEAVE™ probe detection of nucleic acid sequences in a test sample. The probe protector oligonucleotide is designed to be substantially complementary to the FRET pair labeled CATACLEAVE™ oligonucleotide probe. Base pairing between the probe protector oligonucleotide and the CATACLEAVE™ oligonucleotide probe coupled with chemical modification of probe sequences prevents endonucleolytic degradation of RNA sequences in the oligonucleotide probe as well as the formation of spurious RNA:DNA hybrids during PCR cycling that can lead to non-specific cleavage of the oligonucleotide probe prior to target nucleic acid sequence detection.

In certain embodiments, a probe protector oligonucleotide can have the structure of R3-X′—R4 (Formula II) that is substantially complementary to an oligonucleotide probe having the structure of R1-X—R2 (Formula I), wherein each of R1, R2, R3, and R4 is selected from a nucleic acid or a nucleic acid analog and each of X and X′ is a naturally occurring or modified RNA, and wherein the oligonucleotide probe nucleic acid sequence is substantially complementary to a target nucleic acid sequence.

In further embodiments, this disclosure describes a method of stabilizing an oligonucleotide probe comprising the steps of hybridizing a probe protector probe with a oligonucleotide probe, wherein the hybridization stabilizes the oligonucleotide probe.

The method can include the step of storing the stabilized oligonucleotide probe.

The melting temperature, Tm, of the probe protector oligonucleotide hybridizing with the oligonucleotide probe can be lower than the melting temperature, Tm′, of said oligonucleotide probe hybridizing with the target nucleic acid sequence.

In other embodiments, the melting temperature, Tm, of the probe protector oligonucleotide hybridizing with the oligonucleotide probe can be lower by about 10° C. as compared to the melting temperature, Tm′, of the oligonucleotide probe hybridizing to said target nucleic acid sequence.

In one aspect, a protected probe is disclosed where an oligonucleotide probe is base paired to a probe protector oligonucleotide.

In another embodiment, a method for the real-time detection of a target DNA sequence in a sample is described, comprising the steps of providing a sample comprising a target DNA sequence, providing a pair of forward and reverse amplification primers that can anneal to the target DNA sequence, providing a protected probe comprising a labeled oligonucleotide probe base-paired to a protected probe oligonucleotide, wherein the oligonucleotide probe comprises RNA and DNA nucleic acid sequences that are substantially complementary to the target DNA sequence, amplifying a PCR fragment between the forward and reverse amplification primers in the presence of an amplifying polymerase activity, an amplification buffer, and an RNase H activity and the protected probe under conditions where the oligonucleotide probe can dissociate from the protector probe oligonucleotide and the RNA sequences of the oligonucleotide probe can form a RNA:DNA heteroduplex with the target DNA sequence present in the PCR fragment, and detecting a real-time increase in the emission of a signal from the label on the oligonucleotide probe, wherein the increase in signal indicates the presence of the target DNA sequence in the sample.

In yet another embodiment, a method for the real-time detection of a target RNA sequence in a sample is described, comprising the steps of providing a sample comprising a target RNA sequence, providing a pair of forward and reverse amplification primers that can anneal to the target nucleic acid sequence, providing a protected probe comprising a labeled oligonucleotide probe base-paired to a protected probe oligonucleotide, wherein the oligonucleotide probe comprises RNA and DNA nucleic acid sequences that are substantially complementary to the target nucleic acid sequence, reverse transcribing the target RNA in the presence of a reverse transcriptase activity and the reverse amplification primer to produce a target cDNA sequence; amplifying a PCR fragment between the forward and reverse amplification primers in the presence of an amplifying polymerase activity, an amplification buffer, and an RNase H activity and the protected probe under conditions where the oligonucleotide probe can dissociate from the protector probe oligonucleotide and the RNA sequences of the oligonucleotide probe can form a RNA:DNA heteroduplex with the target DNA sequence present in the PCR fragment; and detecting a real-time increase in the emission of a signal from the label on the oligonucleotide probe, wherein the increase in signal indicates the presence of the target RNA sequence in the sample.

In another embodiment, the disclosure provides for a kit for the real-time detection of a target nucleic acid sequences in a sample comprising a probe protector oligonucleotide having the structure of R3-X′—R4 (Formula II) and oligonucleotide probe having the structure of R1-X—R2 (Formula I), wherein the probe protector oligonucleotide is substantially complementary to an oligonucleotide probe, each of R1, R2, R3, and R4 is selected from a nucleic acid or a nucleic acid analog, each of X and X′ is a naturally occurring or modified RNA, and wherein the oligonucleotide probe nucleic acid sequence is substantially complementary to a target nucleic acid sequence.

The kit can include positive internal and negative controls and/or uracil-N-glycosylase, and/or an amplification buffer and/or an amplifying polymerase activity such as a thermostable DNA polymerase and/or a reverse transcriptase activity and/or an RNase H activity such as the enzymatic activity of a thermostable RNase H or a hot start RNase H.

The protected probe can include a labeled oligonucleotide probe having the structure of R1-X—R2 (Formula I) base-paired with a probe protector oligonucleotide having the structure of R3-X′—R4 (Formula II), wherein each of R1, R2, R3, and R4 is selected from a nucleic acid or a nucleic acid analog, each of X and X′ is a naturally occurring or modified RNA, and wherein the probe protector oligonucleotide is substantially complementary to the oligonucleotide probe.

The real-time increase in the emission of the signal from the label on the oligonucleotide probe results from the RNase H cleavage of the heteroduplex formed between the oligonucleotide probe and one of the strands of the PCR fragment.

The amplifying step occurs under conditions where the oligonucleotide probe can dissociate from the protector probe oligonucleotide and the RNA sequences of the oligonucleotide probe can form an RNA:DNA heteroduplex with the target DNA sequence present in the PCR fragment.

The PCR fragment can be amplified by polymerase chain reaction (PCR), rolling circle amplification (RCA), nucleic acid sequence based amplification (NASBA), or strand displacement amplification (SDA).

The oligonucleotide probe can be labeled with an enzyme, an enzyme substrate, a radioactive material, a fluorescent dye, a chromophore, a chemi-luminescence label, an electrochemical luminescence label, or a ligand having a binding partner.

The oligonucleotide probe can be labeled with a fluorescence resonance energy transfer (FRET) pair comprising a fluorescence donor and a fluorescence acceptor.

The fluorescence donor emission of the oligonucleotide probe is quenched by the fluorescence acceptor but cleavage of the RNA sequences of the oligonucleotide probe precludes quenching by the fluorescence acceptor.

The RNase H activity can be the activity of a thermostable RNase H. RNase H activity can be a hot start RNase H activity.

The PCR fragment can be linked to a solid support.

The amplifying polymerase activity can be an activity of a thermostable DNA polymerase.

The nucleic acid within the sample can be pre-treated with uracil-N-glycosylase, that is inactivated prior to PCR amplification.

The amplification buffer can be a Tris-acetate buffer.

The —OH at a 3′ end of the oligonucleotide probe can be blocked to prevent the oligonucleotide probe from being a substrate for primer extension by a template-dependent nucleic acid polymerase.

The melting temperature, Tm, of the probe protector oligonucleotide hybridizing with the oligonucleotide probe can be lower than the melting temperature, Tm′, of said oligonucleotide probe hybridizing with the target nucleic acid sequence.

In other embodiments, the melting temperature, Tm, of the probe protector oligonucleotide hybridizing with the oligonucleotide probe can be lower by about 10° C. as compared to the melting temperature, Tm′, of the oligonucleotide probe hybridizing to said target nucleic acid sequence.

The previously described embodiments have many advantages, including using a protected CATACLEAVE™ to improve the stability of typical Catacleave™ probe which contains instable RNA ribonucleotides. The improved detection method is fast, accurate and suitable for high throughput applications. Convenient, user-friendly and reliable kits are also described for the high throughput detection of target nucleic acid sequences.

### BRIEF DESCRIPTION OF THE DRAWINGS

The skilled artisan will understand that the drawings, described below, are for illustration purposes only. The figures are not intended to limit the scope of the teachings in any way.

FIG. 1 is a schematic representation of CATACLEAVE™ probe technology.

FIG. 2 is a schematic representation of real-time CATACLEAVE™ probe detection of PCR amplification products.

FIG. 3 is a graph showing results of real-time PCR performed with a reaction mixture containing both a protected probe and an unprotected probe;

FIGS. 4 A-C show real-time PCR results after an unprotected probe (FIG. 4A) and a probe protected with the RNA-comp1 (FIG. 4B) or the RNA-comp2 (FIG. 4C) were stored at different temperatures for 24 hours;

FIGS. 5 A-C show real-time PCR results after an unprotected probe (FIG. 5A) and a probe protected with the RNA-comp1 (FIG. 5B) or the RNA-comp2 (FIG. 5C) were stored at different temperatures for 48 hours;

FIGS. 6 A-C show real-time PCR results after unprotected probes and probes protected with the RNA-comp1 or the RNA-comp2 were stored at −20° C. (FIG. 6A), 4° C. (FIG. 6B), and 30° C. (FIG. 6C) for 17 days;

FIGS. 7 A-C show real-time PCR results after an unprotected probe and a probe protected with the RNA-comp1 or the RNA-comp2 were stored at −20° C. (FIG. 7A), 4 r (FIG. 7B), and 30° C. (FIG. 7C) for 30 days, respectively;

FIG. 8 shows the relative intensity change of fluorescent signals of real-time PCR results after a protected probe was stored under different conditions;

FIGS. 9 A-B show real-time PCR results targeting E. coli 0157:H7 sequences using protected (FIG. 9A) and unprotected probes (FIG. 9B) that were stored at −20° C., 4° C., and 30° C. for 40 days;

FIGS. 10 A-B show real-time PCR results targeting Salmonella sequences using protected (FIG. 10A) and unprotected probes (FIG. 10B) that were stored at −20° C., 4° C., and 30° C. for 40 days;

FIGS. 11A-B show real-time PCR results targeting Listeria sequences using protected (FIG. 11A) and unprotected probes (FIG. 11B) that were stored at −20° C., 4° C., and 30° C. for 40 days;

FIGS. 12 A-B show real-time PCR results targeting E. coli 0157:H7 sequences using protected (FIG. 12A) and unprotected probe (FIG. 12B) that were stored at −20° C., 4° C., and 30° C. for 60 days;

FIGS. 13 A-B show real-time PCR results targeting Salmonella sequences using protected (FIG. 13A) and unprotected probe (FIG. 13B) that were stored at −20° C., 4° C., and 30° C. for 60 days;

FIGS. 14 A-B show real-time PCR results targeting Listeria sequences using protected (FIG. 14A) and unprotected probe (FIG. 14B) that were stored at −20° C., 4° C., and 30° C. for 60 days;

FIG. 15 is a graph showing results of real-time PCR performed with a reaction mixture containing both a protected probe and an unprotected probe and 10-10⁶ copies of E. coli O157:H7 as a target nucleic acid sequence.

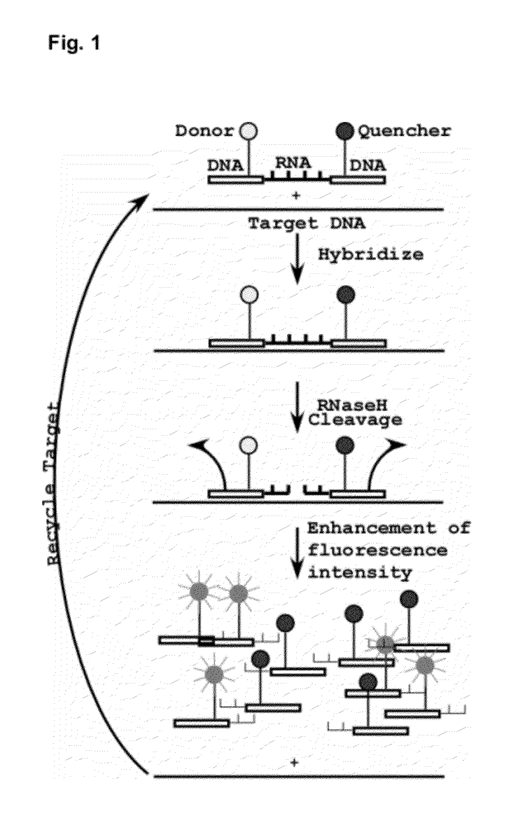

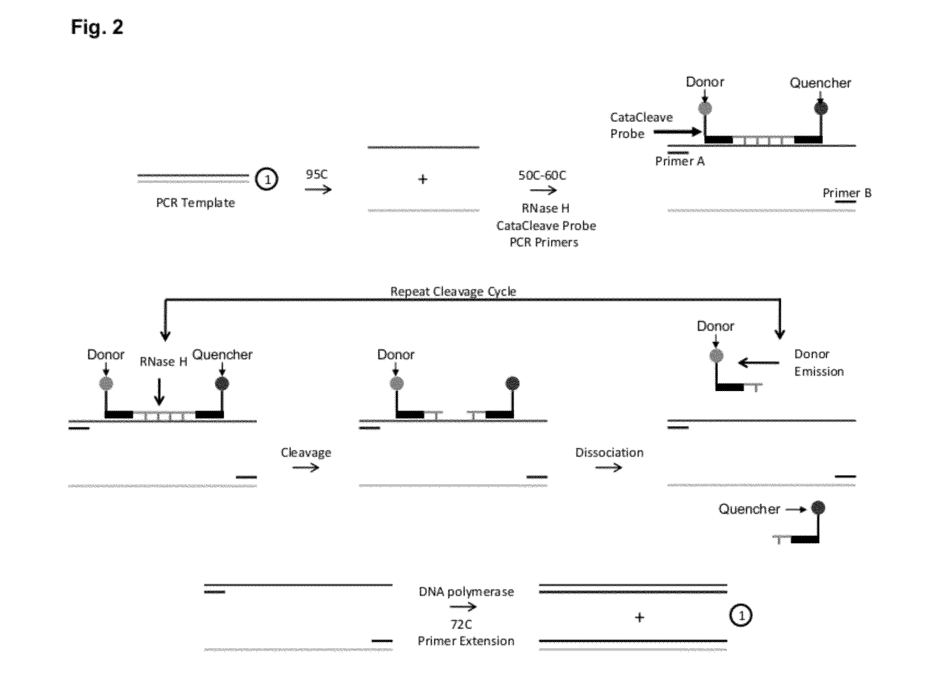

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

The term “nucleic acid analog” used herein refers to a molecule including one or more nucleotide analogs and/or one or more phosphate ester analogs and/or one or more pentose analogs. An example of the nucleic acid analog is a molecule in which a phosphate ester bond and/or a sugar phosphate ester bond is to be substituted with another type of bond, for example, an N-(2-aminoethyl)-glycine amide bond and other amide bonds. Another example of the nucleic acid analog may be a molecule that includes one or more nucleotide analogs and/or one or more phosphate ester analogs and/or one or more pentose analogs and may form a double strand by hybridization between the nucleic acids, the nucleic acid analogs and/or the nucleic acids and the nucleic acid analogs.

A “target DNA or “target RNA” or “target nucleic acid,” or “target nucleic acid sequence” refers to a nucleic acid that is targeted by DNA amplification. A target nucleic acid sequence serves as a template for amplification in a PCR reaction or reverse transcriptase-PCR reaction. Target nucleic acid sequences may include both naturally occurring and synthetic molecules. Exemplary target nucleic acid sequences include, but are not limited to, genomic DNA or genomic RNA.

As used herein, “label” or “detectable label” can refer to any chemical moiety attached to a nucleotide, nucleotide polymer, or nucleic acid binding factor, wherein the attachment may be covalent or non-covalent. Preferably, the label is detectable and renders said nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

The probe protector technology is now described in the context of a CATACLEAVE™ real time PCR detection of target nucleic acid sequences including:
(1) Selection of forward and reverse amplification primer sequences
(2) Optional enrichment for bacterial cells in a test sample;
(3) Nucleic acid template preparation from prokaryotic and eukaryotic cells;
(4) PCR amplification of target nucleic acid sequences;
(5) Optional reverse transcriptase-PCR amplification of a RNA target nucleic acid sequence;
(6) Real-time PCR using a CATACLEAVE™ probe;
(7) Labeling of a CATACLEAVE™ probe;
(8) Attachment of a CATACLEAVE™ probe to a solid support;
(9) RNase H cleavage of the CATACLEAVE™ Probe;
(10) Probe protector oligonucleotide;
(11) CATACLEAVE™ real-time PCR using stabilized CATACLEAVE™ oligonucleotide probes, and
(12) Kits

### Selection of Primer Sequences

A person of skill in the art will know how to design PCR primers flanking a target nucleic acid sequence of interest. Synthesized oligos are typically between 20 and 26 base pairs in length with a melting temperature, Tm, of about 55 degrees.

In certain embodiments, primer sequences are selected for their inability to form primer dimers in a standard PCR reaction. A “primer dimer” is a potential by-product in PCR, that consists of primer molecules that have partially hybridized to each other because of strings of complementary bases in the primers. As a result, the DNA polymerase amplifies the primer dimer, leading to competition for PCR reagents, thus potentially inhibiting amplification of the DNA sequence targeted for PCR amplification. In real-time PCR, primer dimers may interfere with accurate quantification by reducing sensitivity. Primer pairs are therefore selected according to their ability not to form primer dimers during PCR amplification. Such primers are capable of detecting single target molecules in as little as about 40 PCR cycles using optimum amplification conditions.

### Optional Enrichment for Bacterial Cells in a Test Sample

An optional protocol for detecting target prokaryotic sequences may include the steps of providing a food sample or surface wipe, mixing the sample or wipe with a growth medium and incubating to increase the number or population of microorganisms (“enrichment”), disintegrating the cells (“lysis”), and subjecting the obtained lysate to amplification and detection of target nucleic acid sequences. Food samples may include, but are not limited to, fish such as salmon, dairy products such as milk, and eggs, poultry, fruit juices, meats such as ground pork, pork, ground beef, or beef, vegetables such as spinach or alfalfa sprouts, or processed nuts such as peanut butter.

### Nucleic Acid Template Preparation

Procedures for the extraction and purification of a nucleic acid template from samples are well known in the art and are described in, for example, Chapter 2 (DNA) and Chapter 4 (RNA) of F. Ausubel et al., eds., Current Protocols in Molecular Biology, Wiley-Interscience, New York (1993).

For DNA, these protocols generally entail gently lysing the cells with solubilization of the DNA and enzymatically or chemically substantially freeing the DNA from contaminating substances such as proteins, RNA and other substances (i.e., reducing the concentrations of these contaminants in the same solution as the DNA to a level that is low enough that the molecular biological procedures of interest can be carried out).

RNA isolation methods have used liquid-liquid extraction (i.e, phenol-chloroform) and alcohol precipitation. Perhaps, the most commonly used liquid-liquid extraction method is the “acid-guanidinium-phenol” method of Chomczynski and Sacchi (Chomczynski P, Sacchi N., Single-step method of RNA isolation by acid guanidinium thiocyanate-phenol-chloroform extraction, Anal Biochem 162: 156-9 [1987]; U.S. Pat. Nos. 5,945,515, 5,346,994, and 4,843,155).

More recently, various solid phase purification protocols have been developed where the nucleic acid is bound to a solid support while impurities such as proteins, and phospholipids are selectively eluted.

Solid phase methods can be classified broadly according to the type of solid phase used for such extractions, either silica or ion-exchange resins. For solid phase nucleic acid isolation methods, many solid supports have been used including membrane filters, magnetic beads, metal oxides, and latex particles. Probably the most widely used solid supports are silica-based particles (see, e.g., U.S. Pat. No. 5,234,809 (Boom et al.); International Publication No. WO 95/01359 (Colpan et al.);U.S. Pat. No. 5,405,951 (Woodard); International Publication No. WO 95/02049 (Jones); WO 92/07863 (Qiagen GmbH). Nucleic acids bind to silica in the presence of chaotropic agents.

A number of commercial kits are also available for high throughput nucleic acid isolation (e.g. MagMAX™ RNA Isolation Kits, Ambion Cat. # AM1830; Wizard® SV 96 Genomic DNA Purification System, Promega, Cat. # A6780).

In preferred embodiments, the sample to be tested for target nucleic acid sequences is a cell lysate that does not require additional purification prior to real time PCR. Here again, a number of commercial kits are available for example, TaqMan® Gene Expression Cells-to-CTT™ Kit (Ambion, Cat. # AM1728)

Cells also can be lysed cells using a lysis buffer having a pH of about 6 to about 9, a zwitterionic detergent at a concentration of about 0.125% to about 2%, an azide at a concentration of about 0.3 to about 2.5 mg/ml and a protease such as proteinase K (about 1 mg/ml). After incubation at 55° C. for 15 minutes, the proteinase K is inactivated at 95° C. for 10 minutes to produce a “substantially protein free” lysate that is compatible with high efficiency PCR or reverse transcription PCR analysis.

In one embodiment, the 1× lysis reagent contains 12.5 mM Tris acetate or Tris-HCl or HEPES (4-(2-hydroxyethyl)-1-piperazineethanesulfonic acid) (pH=7-8), 0.25% (w/v) CHAPS, 0.3125 mg/ml sodium azide and proteinase K at 1 mg/ml.

The term “lysate” as used herein, refers to a liquid phase with lysed cell debris and nucleic acids.

As used herein, the term “substantially protein free” refers to a lysate where most proteins are inactivated by proteolytic cleavage by a protease. Protease may include proteinase K. Addition of proteinase K during cell lysis rapidly inactivates nucleases that might otherwise degrade the target nucleic acids. The “substantially protein free” lysate may be or may not be subjected to a treatment to remove inactivated proteins.

For the lysis of gram negative bacteria, such as Salmonella, Listeria and E. coli, proteinase K to 1 mg/ml may be added to the lysis reagent. After incubation at 55° C. for 15 minutes, the proteinase K is inactivated at 95° C. for 10 minutes to produce a substantially protein free lysate that is compatible with high efficiency PCR or reverse transcription PCR analysis.

As used herein, “zwitterionic detergent” refers to detergents exhibiting zwitterionic character (e.g., does not possess a net charge, lacks conductivity and electrophoretic mobility, does not bind ion-exchange resins, breaks protein-protein interactions), including, but not limited to, CHAPS, CHAPSO and betaine derivatives, e.g. preferably sulfobetaines sold under the brand names Zwittergent® (Calbiochem, San Diego, Calif.) and Anzergent® (Anatrace, Inc. Maumee, Ohio).

In one embodiment, the zwitterionic detergent is CHAPS (CAS Number: 75621-03-3; available from SIGMA-ALDRICH product no. C3023-1G), an abbreviation for 3-[(3-cholamidopropyl) dimethylammonio]-1-propanesulfonate (described in further detail in U.S. Pat. No. 4,372,888) having the structure:

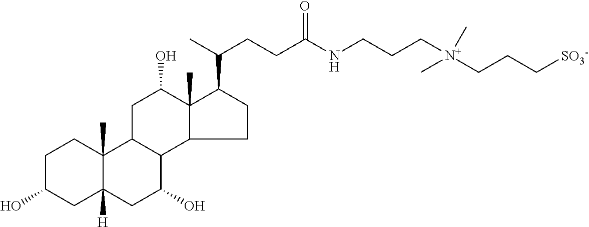

In a further embodiment, CHAPS is present at a concentration of about 0.125% to about 2% weight/volume (w/v) of the total composition. In a further embodiment, CHAPS is present at a concentration of about 0.25% to about 1% w/v of the total composition. In yet another embodiment, CHAPS is present at a concentration of about 0.4% to about 0.7% w/v of the total composition.

In other embodiments, the lysis buffer may include other non-ionic detergents such as Nonidet, Tween or Triton X-100.

As used herein, the term “lysis buffer” refers to a composition that can effectively maintain the pH value between 6 and 9, with a pKa at 25° C. of about 6 to about 9. The buffer described herein is generally a physiologically compatible buffer that is compatible with the function of enzyme activities and enables biological macromolecules to retain their normal physiological and biochemical functions.

Examples of buffers added to a lysis buffer include, but are not limited to, HEPES ((4-(2-hydroxyethyl)-1-piperazineethanesulfonic acid), MOPS (3-(N-morpholino)-propanesulfonic acid), N-tris(hydroxymethyl)methylglycine acid (Tricine), tris(hydroxymethyl)methylamine acid (Tris), piperazine-N,N′-bis(2-ethanesulfonic acid) (PIPES) and acetate or phosphate containing buffers (K₂HPO₄, KH₂PO₄, Na₂HPO₄, NaH₂PO₄) and the like.

The term “azide” as used herein is represented by the formula —N3. In one embodiment, the azide is sodium azide NaN₃ (CAS number 26628-22-8; available from SIGMA-ALDRICH Product number: S2002-25G) that acts as a general bacterioside.

The term “protease,” as used herein, is an enzyme that hydrolyses peptide bonds (has protease activity). Proteases are also called, e.g., peptidases, proteinases, peptide hydrolases, or proteolytic enzymes. The proteases for use according to the invention can be of the endo-type that act internally in polypeptide chains (endopeptidases). In one embodiment, the protease can be the serine protease, proteinase K (EC 3.4.21.64; available from Roche Applied Sciences, recombinant proteinase K 50 U/ml (from Pichia pastoris) Cat. No. 03 115 887 001).

Proteinase K is used to digest protein and remove contamination from preparations of nucleic acid. Addition of proteinase K to nucleic acid preparations rapidly inactivates nucleases that might otherwise degrade the DNA or RNA during purification. It is highly-suited to this application since the enzyme is active in the presence of chemicals that denature proteins and it can be inactivated at temperatures of about 95° C. for about 10 minutes.

In addition to or in lieu of proteinase K, the lysis reagent can comprise a serine protease such as trypsin, chymotrypsin, elastase, subtilisin, streptogrisin, thermitase, aqualysin, plasmin, cucumisin, or carboxypeptidase A, D, C, or Y. In addition to a serine protease, the lysis solution can comprise a cysteine protease such as papain, calpain, or clostripain; an acid protease such as pepsin, chymosin, or cathepsin; or a metalloprotease such as pronase, thermolysin, collagenase, dispase, an aminopeptidase or carboxypeptidase A, B, E/H, M, T, or U. Proteinase K is stable over a wide pH range (pH 4.0-10.0) and is stable in buffers with zwitterionic detergents.

### PCR Amplification of Target Nucleic Acid Sequences

Once the primers are prepared, nucleic acid amplification can be accomplished by a variety of methods, including, but not limited to, the polymerase chain reaction (PCR), nucleic acid sequence based amplification (NASBA), ligase chain reaction (LCR), and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. An PCR fragment is typically, but not exclusively, a DNA PCR fragment. A PCR fragment can be single-stranded or double-stranded, or in a mixture thereof in any concentration ratio. A PCR fragment or RT-PCT can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino)propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 μM each of nucleotides dATP, dCTP, dGTP and dTTP. The buffers of the invention may contain additives to optimize efficient reverse transcriptase-PCR or PCR reaction.

The term “nucleotide,” as used herein, refers to a compound comprising a nucleotide base linked to the C-1′ carbon of a sugar, such as ribose, arabinose, xylose, and pyranose, and sugar analogs thereof. The term “nucleotide” includes a ribonucleoside triphosphate such as rATP, rCTP, rGTP, or rUTP, and a deoxyribonucleoside triphosphate such as dATP, dCTP, dGTP, or dTTP.

The term “nucleoside” used herein refers to a combination of a base and a sugar, that is, a nucleotide that does not include a phosphate moiety. The term “nucleoside” and the term “nucleotide” may also be used inter-changeably in the art. For example, dUTP is deoxyribonucleoside triphosphate, and when inserted into DNA, may act as a DNA monomer, that is, dUMP or deoxyuridin monophosphate. In this regard, even when obtained DNA does not include dUTP, it can be said that dUTP is inserted into DNA.

The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499).

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, an “amplifying polymerase activity” refers to an enzymatic activity that catalyzes the polymerization of deoxyribonucleotides. Generally, the enzyme will initiate synthesis at the 3′-end of the primer annealed to a nucleic acid template sequence, and will proceed toward the 5′ end of the template strand. In certain embodiments, an “amplifying polymerase activity” is a thermostable DNA polymerase.

As used herein, a thermostable polymerase is an enzyme that is relatively stable to heat and eliminates the need to add enzyme prior to each PCR cycle.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) i (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LApro Taq™, and EX Taq™. In another embodiment, the thermostable polymerase used in the multiplex amplification reaction of the invention is the AmpliTaq Stoffel fragment.

The nucleic acid polymerase may have a concentration of 0.1 unit/μl or more in a reaction mixture. For example, the concentration of the nucleic acid polymerase in the reaction mixture may be in the range of 0.1 to 10 unit/μl, 0.1 to 5 unit/μl, 0.1 to 2.5 unit/μl, or 0.1 to about 1 unit/μl.

The amplifying can be performed by using, for example, an amplification method selected from the group consisting of polymerase chain reaction (PCR), rolling circle amplification (RCA), nucleic acid sequence based amplification (NASBA), and strand displacement amplification (SDA). The hybridization means formation of a duplex by complementarily linking strands of a 2-stranded nucleic acid. The hybridization may be performed by using any known method in the art. For example, the hybridization may be performed by separating a duplex into single strands by heating a primer and/or a target sequence and cooling to allow two complementary strands to be linked. If the target sequence is a single strand, the separation of the primer and/or the target sequence may not be needed. The hybridization may be performed using a buffer that is appropriate for the kind of the selected primer and/or target sequence selected, for example, a buffer with an appropriate salt concentration and an appropriate pH. The extension is well known in the art. The extension may be performed by using, for example, a DNA polymerase, a RNA polymerase, or a reverse transcriptase. The nucleic acid polymerase may be thermally stable, for example, may retain its activity when exposed to a temperature of 95° C. or more. A thermostable DNA polymerase may be an enzyme separated from a thermophilic bacteria as defined herein. For example, the thermally stable DNA polymerase may be a Taq polymerase having an optimal activity at a temperature of about 70° C.

### Reverse Transcriptase-PCR Amplification of a RNA Target Nucleic Acid Sequence

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise measurement of RNA sequences with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan™”) or endonuclease assay (“CATACLEAVE™”), discussed below.

### Real-Time PCR Using a CATACLEAVE™ Probe

Post-amplification amplicon detection can be both laborious and time consuming. Real-time methods have been developed to monitor amplification during the PCR process. These methods typically employ fluorescently labeled probes that anneal to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan™ probes (e.g., U.S. Pat. Nos. 5,210,015 and 5,487,972), and CATACLEAVE™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CATACLEAVE™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CATACLEAVE™”). CATACLEAVE™ technology differs from TaqMan™ in that cleavage of the probe is accomplished by a second enzyme that does not have polymerase activity. The CATACLEAVE™ probe has a sequence within the molecule which is a target of an endonuclease, such as, for example a restriction enzyme or RNAase. In one example, the CATACLEAVE™ probe has a chimeric structure where the 5′ and 3′ ends of the probe are constructed of DNA and the cleavage site contains RNA.

For example, the probe may have a structure represented by Formula I below:

R1-X—R2 (Formula I)

wherein R1 and R2 are each selected from the group consisting of a nucleic acid and a nucleic acid analog, and X may be a first RNA. For example, R1 and R2 may all be DNA; R1 may be DNA and R2 may be RNA; R1 may be RNA and R2 may be DNA; or R1 and R2 may all be RNA. The nucleic acid or nucleic acid analog of R1 and R2 may be a protected nucleic acid. For example, the nucleic acid and the nucleic acid analog may be methylated and thus, may be resistant to decomposition due to an RNA specific decomposition enzyme (for example, RNase H). A length of the probe may differ according to a target sequence and a PCR condition. An annealing temperature (Tm) of the probe may be about 60° C. or more, about 70° C. or more, or about 80° C. or more.

The probe may be modified. For example, in the probe, a base may be partially or entirely methylated. Such modification of a base may protect the probe from decomposition by an enzyme, a chemical factor, or other factors. In addition, in the probe, —OH at a 5′ end or 3′ end may be blocked. For example, —OH at the 3′ end of the probe may be blocked and thus, the probe may not be a substrate for primer extension by the template-dependent nucleic acid polymerase.

The DNA sequence portions of the probe can be labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CATACLEAVE™ binding. In this way it is possible for a single amplicon to serve as a target or multiple rounds of probe cleavage until the primer is extended through the CATACLEAVE™ probe binding site.

### Labeling of a CATACLEAVE Probe

The term “probe” (also called “oligonucleotide probe” or “CATACLEAVE™ probe”) comprises a polynucleotide that comprises a specific portion designed to hybridize in a sequence-specific manner with a complementary region of a specific nucleic acid sequence, e.g., a target nucleic acid sequence. A length of the probe may be in the range of, for example, about 10 to about 200 nucleotides, about 15 to about 200 nucleotides, or about 15 to about 60 nucleotides in length, more preferably, about 18 to about 30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CATACLEAVE™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, “label” or “detectable label” of the CATACLEAVE probe refers to any moiety that is detectable by using a spectroscopic, photo-chemical, biochemical, immunochemical, or chemical method. The detectable label may be selected from the group consisting of an enzyme, an enzyme substrate, a radioactive material, a fluorescent dye, a chromophore, a chemi-luminescence label, an electrochemical luminescence label, a ligand having a particular bonding partner, and other labels that interact with each other to increase, change, or reduce a signal. The detectable label may survive during heat cycling of a PCR.

The detectable label may be a fluorescence resonance energy transfer (FRET) pair. The detectable label may be a FRET pair, and a fluorescence donor and a fluorescence receptor may be spaced apart from each other at an appropriate interval and thus, fluorescence donor emission is hindered and is activated by disassociation caused by cleaving. That is, in the probe, when the probe is not cleaved, a fluorescence donor emission is quenched by a fluorescence acceptor emission by FRET between two chromophores. When a donor chromophore is located near the acceptor chromophore, a donor chromophore in an excited state may transfer energy to an acceptor chromophore. The transfer is always non-radiative and may occur by dipole-dipole coupling. If the distance between two chromophores is sufficiently increased, FRET efficiency is decreased and the donor chromophore emission may be radiatively detected.

In one embodiment, the detectable label can be a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs light emitted from the fluorescence donor. The second fluorochrome absorbs the light that is emitted from the fluorescence donor and emits light of longer wavelength than the light emitted by the fluorescence donor. The quenching molecule absorbs light emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl) maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a CATACLEAVE™ Probe to a Solid Support

In one embodiment, the oligonucleotide probe can be present as a free form in a solution or attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are complementary to a selected region of a target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe, wherein the increase in signal indicates the presence of the polymorphism in the target DNA.

### RNase H Cleavage of the Catacleave™ Probe

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase H II enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108).

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus RNase HII, Pyrococcus horikoshi RNase HII, Thermococcus litoralis RNase HI, Thermus thermophilus RNase HI.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase HII (SEQ ID NO: 1), shown below.

| (SEQ ID NO: 1) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997)

In another embodiment, an RNase H enzyme is a thermostable RNase H with at least one or more homology regions 1-4 corresponding to positions 5-20, 33-44, 132-150, and 158-173 of SEQ ID NO: 1.

HOMOLOGY REGION 1:
GIDEAG RGPAIGPLVV (SEQ ID NO: 19;
corresponding to positions 5-20 of SEQ ID NO: 1)
HOMOLOGY REGION 2:
LRNIGVKD SKQL (SEQ ID NO: 20;
corresponding to positions 33-44 of SEQ ID NO: 1)
HOMOLOGY REGION 3:
HKADAKYPV VSAASILAKV (SEQ ID NO: 21;
corresponding to positions 132-150 of SEQ ID NO: 1)
HOMOLOGY REGION 4:
KLK KQYGDFGSGY PSD (SEQ ID NO: 22;
corresponding to positions 158-173 of SEQ ID NO: 1)

In another embodiment, an RNase H enzyme is a thermostable RNase H with at least one of the homology regions having 50%, 60%. 70%, 80%, 90%, 95% sequence identity with a polypeptide sequence of SEQ ID NOs: 19, 20, 21 and 22.

The terms “sequence identity” as used herein refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiment, the ligand may induce a conformational change. The chemical modification can be a crosslinking (for example, by formaldehyde) or acylation. The release or decoupling of the inhibiting factor from the RNase HII may be accomplished by heating a sample or a mixture containing the coupled RNase HII (inactive) to a temperature of about 65° C. to about 95° C. or higher, and/or lowering the pH of the mixture or sample to about 7.0 or lower.

As used herein, a hot start “inducible” RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Crosslinking of RNase H enzymes can be performed using, for example, formaldehyde. In one embodiment, a thermostable RNase HII is subjected to controlled and limited crosslinking using formaldehyde. By heating an amplification reaction composition, which comprises the modified RNase HII in an active state, to a temperature of about 95° C. or higher for an extended time, for example about 15 minutes, the crosslinking is reversed and the RNase HII activity is restored.

In general, the lower the degree of crosslinking, the higher the endonuclease activity of the enzyme is after reversal of crosslinking. The degree of crosslinking may be controlled by varying the concentration of formaldehyde and the duration of crosslinking reaction. For example, about 0.2% (w/v), about 0.4% (w/v), about 0.6% (w/v), or about 0.8% (w/v) of formaldehyde may be used to crosslink an RNase H enzyme. About 10 minutes of crosslinking reaction using 0.6% formaldehyde may be sufficient to inactivate RNase HII from Pyrococcus furiosus.

The crosslinked RNase HII does not show any measurable endonuclease activity at about 37° C. In some cases, a measurable partial reactivation of the crosslinked RNase HII may occur at a temperature of around 50° C., which is lower than the PCR denaturation temperature. To avoid such unintended reactivation of the enzyme, it may be required to store or keep the modified RNase HII at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds-4 minutes is sufficient to restore RNase H activity. In one embodiment, using a commercially available buffer such as Invitrogen AgPath™ buffer, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Probe Protector Oligonucleotide

The CATACLEAVE™ oligonucleotide probe as disclosed herein can be a single-stranded oligonucleotide that includes RNA and DNA sequences, the ends of which can be labeled with a FRET pair comprising a fluorescence donor and quencher. Endonucleolytic cleavage of the oligonucleotide probe results in the separation of the fluorescence donor from the quencher and an increase in the emission of fluorescence.

Any spurious cleavage of CATACLEAVE™ oligonucleotide probe prior to target nucleic acid sequence detection can therefore lead to an increase in the level of background non-specific fluorescence that compromises the overall sensitivity of the assay. Such premature cleavage of the CATACLEAVE™ oligonucleotide probe can have several causes. For example, the sample can be a cell lysate that may have residual contaminating nucleases that can cleave single-stranded RNA or DNA. Single stranded sequence may be susceptible to changes in pH or the presence of free radicals. During PCR cycling, the RNA moiety of the CATACLEAVE™ oligonucleotide probe may be able to form non-specific RNA:DNA heteroduplexes that may be cleaved by the RNase H in the amplification buffer. Whatever the underlying cause, the CATACLEAVE™ oligonucleotide probe is highly susceptible to endonucleolytic cleavage either during the real time PCR reaction or during long term storage.

To offset these potential problems, this disclosure provides for a probe protector oligonucleotide (also referred to throughout the specification as “complementary strand” or “protector” or “probe protector”), which is at least partially complementary to the “oligonucleotide probe” (also referred to throughout the specification as a “probe” or “CATACLEAVE™ probe”). The probe protector oligonucleotide, which can also be modified by methylation, is therefore designed to base pair with the CATACLEAVE™ oligonucleotide probe and “protect” it from degradation or non-specific hybridization with DNA sequences during real-time PCR cycling.

Thus, in an astutely planned amplification protocol, the single-stranded CATACLEAVE™ oligonucleotide probe may only be exposed just prior to annealing with the selected target DNA sequence in the amplified PCR fragment, producing an RNA:DNA heteroduplex that can be cleaved by RNase H in the amplification mix resulting in a concomitant increase in the emission of fluorescence.

The oligonucleotide probe and the probe protector oligonucleotide are each represented by Formula I and Formula II, respectively:

R1-X—R2 (Formula I)

R3-X′—R4 (Formula II)

wherein each of R1 and R2 is selected from a nucleic acid or a nucleic acid analog and X is an RNA. The X sequence of the probe is complementary to the X′ sequence of the probe protector oligonucleotide, thus, the first probe and the second probe can base pair with each other.

In Formula I and Formula II, at least one of R1, R2, R3 and R4 may be a nucleic acid or a nucleic acid analog.

For example, R1 and R2 may be DNA; R1 may be DNA and R2 may be RNA; R1 may be RNA and R2 may be DNA; or R1 and R2 may be both RNA.

For example, R3 and R4 may be both DNA; R3 may be DNA and R4 may be RNA; R3 may be RNA and R4 may be DNA; or R3 and R4 may be both RNA.

In one embodiment, X can be at least partially complementary to X′. In another embodiment, X is fully complementary to X′.

The nucleic acid or nucleic acid analog of R1, R2, R3 and R4 may be a protected nucleic acid. The nucleic acid and the nucleic acid analog may be modified, for example, methylated, and thus, rendered resistant to enzymatic degradation.

The length of the nucleic acid probe can differ according to the selected target nucleic acid sequence and PCR conditions.

The melting temperature (Tm) of the oligonucleotide probe with respect to a target nucleic acid sequence can be about 50° C. or more, about 60° C. or more, about 70° C. or more, or about 80° C. or more.

The length of the oligonucleotide probe can be in the range of, for example, about 10 to about 200 nucleotides (nt), about 15 to about 200 nt, about 10 to about 60 nt, or about 15 to about 60 nt.

The melting temperature (Tm) of the probe protector oligonucleotide hybridized to the oligonucleotide probe may be lower than the melting temperature (Tm′) of the PCR reaction by, for example, about 5° C. or about 10° C. or about 15° C. or about 20° C. or about 25° C.

For example, the melting temperature (Tm) of the probe protector oligonucleotide hybridized to the oligonucleotide probe may be about 50° C. or less, about 40° C. or less, or about 30° C. or less.

The melting temperature (Tm) of the probe protector oligonucleotide hybridized to the oligonucleotide probe can be about 5° C. or 10° C. or about 15° C. or about 20° C. or about 25° C. lower than the melting temperature (Tm′) of the oligonucleotide probe hybridized to the target nucleic acid sequence.

The oligonucleotide probe or probe protector oligonucleotide may also be modified. For example, one or more bases of the oligonucleotide probe or probe protector oligonucleotide can be partially or entirely methylated. Due to this modification, enzymatic degradation or degradation by other factors can be avoided.

In other embodiments, the —OH at a 5′ end or 3′ end of the oligonucleotide probe or probe protector oligonucleotide may be blocked. For example, —OH at the 3′ end of the oligonucleotide probe or probe protector oligonucleotide may be blocked thereby precluding primer extension from the 3′ end by a template-dependent nucleic acid polymerase.

In certain embodiments, a probe protector may be comprised of two or more oligoncucleotides that may or may not be covalently linked. A probe protector can have the general structure of R1-X—R2 where R1 and R2 are DNA sequences and X is an RNA sequence. In one embodiment, the probe protector may comprise two or more oligonucleotides, for example, an oligonucleotide comprising R1-X sequences and an oligonucleotide comprising the R2 sequence. In another embodiment, the probe protector may comprise an oligonucleotide comprising an R1 sequence and an oligonucleotide comprising an X—R2 sequence. In yet another embodiment, a probe protector may comprise three oligonucleotides having the sequence of R1, R2 and X. In other embodiments, R1, X or R2 sequences may be covalently linked by phosphodiester bonds. In other embodiments, R1, X or R2 sequences may be linked by non covalent means.

### Real-Time Detection of Target Nucleic Acid Sequences Using a Protected CATACLEAVE™ Oligonucleotide Probe

A CATACLEAVE™ oligonucleotide probe is first synthesized with DNA and RNA sequences that are complimentary to a selected target nucleic acid sequence. The probe can be labeled, for example, with a FRET pair, for example, a fluorescein molecule at one end of the probe and a rhodamine quencher molecule at the other end. The probe can be synthesized to be substantially complementary to a target nucleic acid sequence.

A probe protector molecule is synthesized that is substantially complementary to the CATACLEAVE™ oligonucleotide probe. Addition of the probe protector oligonucleotide to a solution of the labeled CATACLEAVE™ oligonucleotide probe therefore results in base pairing between the two molecules to form a substantially double stranded hybrid molecule.

The CATACLEAVE™ oligonucleotide probe and the probe protector oligonucleotide are represented by Formula I and Formula II, respectively:

R1-X—R2 (Formula I)

R3-X—R4 (Formula II)

wherein each of R1, R2, R3, and R4 is selected from a nucleic acid or a nucleic acid analog and X is an RNA which is at least partially hybridize to X′ in Formula II.

The melting temperature (Tm) of the probe protector oligonucleotide with respect to the oligonucleotide can be designed to be lower than melting temperature (Tm′) of the oligonucleotide probe with respect to a target sequence perfectly complementary to the nucleic acid probe. For example, the melting temperature (Tm′) of the probe protector oligonucleotide with respect to the oligonucleotide probe may be lower by about 10° C. compared to the melting temperature (Tm) of the oligonucleotide probe with respect to a target nucleic acid sequence.

At room temperature, the probe protector oligonucleotide is hybridized to the CATACLEAVE™ oligonucleotide probe.

Real-time PCR reagents are then added to a suitable receptacle including a sample comprising a target nucleic acid sequence, forward and reverse amplification primers that can anneal to the target nucleic acid sequence, the protected FRET labeled CATACLEAVE™ oligonucleotide probe, an amplification buffer comprising nucleotides, thermostable DNA polymerase, reverse transcriptase (where applicable) and hot start thermostable RNase H.

Real-time nucleic acid amplification is then performed on the target polynucleotide in the presence of a thermostable nucleic acid polymerases, a thermostable modified RNase H activity, a pair of PCR amplification primers capable of hybridizing to the target polynucleotide, and a labeled CataCleave oligonucleotide probe. For the detection of a target RNA sequence, the reaction mix includes a reverse transcriptase activity for an initial cDNA synthesis step as described herein. During the real-time PCR reaction, by subjecting the mix to a temperature above Tm but below Tm′, the FRET labeled CATACLEAVE™ oligonucleotide probe can dissociate from the probe protector oligonucleotide and anneal the target nucleic acid sequence within the PCR amplicons to form a RNA:DNA heteroduplex that can be cleaved by an RNase H activity. Cleavage of the probe by RNase H leads to the separation of the fluorescent donor from the fluorescent quencher and results in the real-time increase in fluorescence of the probe corresponding to the real-time detection of the target DNA sequences in the sample.

In certain embodiments, the real-time nucleic acid amplification permits the real-time detection of a single target DNA molecule in less than about 40 PCR amplification cycles.

### Storage of Protected CATACLEAVE™ Oligonucleotide Probe

The stabilizing method may include storing of the protected CATACLEAVE™ oligonucleotide probe. The storing of the hybrid molecule may be performed at room temperature or lower. The storage temperature may be, for example, about 40° C. or less, about 30° C. or less, about 20° C. or less, or about 4° C. or less. In another embodiment, the storage temperature may also be 0° C. or less, for example, −4° C. or −20° C. The storage may be maintained for a given period of time. The storage time period may be more than 1 days, for example, 2 days, 17 days, 30 days, or 1 year. The storing may be performed in an alkali condition, for example, at a pH of about 7.0 to about 8.0. In another embodiment, the storage pH may also be acidic, for example, at a pH of about 5.0 to 7.0.

### Kits

The disclosure herein also provides a kit format which comprises a package unit having one or more reagents for the real-time detection of target nucleic acid sequences in a sample using a protected FRET labeled CATACLEAVE™ oligonucleotide probe. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a thermostable polymerase, RNase H, forward and reverse amplification primers, FRET labeled CATACLEAVE™ oligonucleotide probes that can anneal to the real-time PCR products and allow for the detection of the target nucleic acid sequences according to the methodology described herein. In another embodiment, the kit reagents further comprise reagents for the extraction of total genomic DNA, total RNA or polyA⁺ RNA from a sample. Kit reagents may also include reagents for reverse transcriptase-PCR analysis where applicable.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The following examples set forth methods for using the modified RNase H enzyme composition according to the present invention. It is understood that the steps of the methods described in these examples are not intended to be limiting. Further objectives and advantages of the present invention other than those set forth above will become apparent from the examples which are not intended to limit the scope of the present invention.

In the Examples and Figures, the term “protected probe” or “protected” means that the probe (also called or “nucleic acid probe” or “oligonucleotide probe” of Formula I) is hybridized to probe protector oligonucleotide (also called protector or complementary strand) of Formula II.

### Example 1

### Stabilization of Nucleic Acid Probe by Complementary RNA

In this experiment, an oligonucleotide probe having a structure of DNA-RNA-DNA was hybridized with a complementary strand including a sequence complementary to the RNA region and the obtained hybridization product was stored under various conditions. By doing so, it was identified that the nucleic acid probe was stabilized. The nucleic acid probe and the complementary strand used in the experiment are shown in Table 1.

TABLE 1

| | | | SEQ |
|---|---|---|---|
| | Tm | Sequence | ID |
| Name | (° C.) | (5′ → 3′ direction) | NO: |
| Lmon- | 65.3 | CGAATGTAA( [symbol] )rAGACACGGTCTCA | 2 |
| probe | | | |
| RNA-comp1 | 46.7* | (ACCGUGUCU [symbol] UUACAUU)r | 3 |
| RNA-comp2 | 26.3* | (CGUGUCU [symbol] UU)r | 4 |

In Table 1, Lmon-probe is a nucleic acid probe specific to the inlA gene of Listeria monocytogenes, and each of RNA-comp1 and RNA-comp2, which are complementary strands of Formula II, is an RNA including a region (X′) complementary to the RNA region (X in Formula I) of the nucleic acid probe and RNA-comp1 and RNA-comp2 have different lengths. In addition, in Table 1, * represents an annealing temperature (Tm) at which the corresponding RNA is bound to a DNA template, and r represents a ribonucleic acid. The RNA-comp1 and the RNA-comp2 was designed to have short lengths so that Tm may be lower than 50° C. and they may be hybridized to the probe at room temperature or lower, while in general, in a PCR condition in which annealing and extension reactions occur at a temperature of about 60° C., the RNA-comp1 and the RNA-comp2 may be separated from the nucleic acid probe.

The nucleic acid probe (10 mM) and the RNA-comp1 (10 mM) or RNA-comp2 (10 mM) were mixed in the same volume and incubated at a temperature of 65° C. for 5 minutes, and then cooled on ice for 2 minutes or more, thereby hybridizing the RNA-comp1 or the RNA-comp2 to the nucleic acid probe. Then, the protected nucleic acid probe, that is, the double-stranded nucleic acid probe in which the RNA-comp1 or the RNA-comp2 was hybridized was used as a probe for PCR. The probe was labeled with FAM at the 5′ end and with Iowa Black FQ quencher at 3′ end.

A composition of a PCR mixture and PCR conditions are shown in Tables 2 and 3 below.

TABLE 2
PCR mixture composition per each well (μl)

| Component | Volume |
|---|---|
| 10xICAN | 2.5 |
| Forward primer (20 μM) | 1 |
| Reverse primer (20 μM) | 1 |
| Probe (5 μM), protected or unprotected | 1 |
| Fermentas dNTP/dUTP mix ( 2/4 mM) | 1 |
| Taq polymerase 5 u/μl | 0.5 |
| UNG 10 u/μl | 0.1 |
| pfu RNase HII 5 u/μl | 0.2 |
| Template DNA 10⁵ copies/μl | 2 |
| Water | 15.70 |

In Table 1, the template DNA was a plasmid DNA (concentration of 1×10⁵/μl) including Listeria cytomonogenes 23s RNA). Forward primer: Lmon_C3_F:ACGAGTAACGGGACAAATGC (SEQ ID NO: 5), reverse primer: Lmon_C3_R:TCCCTAATCTATCCGCCTGA (SEQ ID NO: 6).

TABLE 3
PCR conditions

| | Temperature | | |
|---|---|---|---|
| Step | (° C.) | Time (min) | Cycle |
| UNG incubation | 37 | 10 | 1 |
| Denaturalization | 95 | 10 | 1 |
| Repetition | 95 | 0.25 | 50 |
| | 60 | 0.33 | |

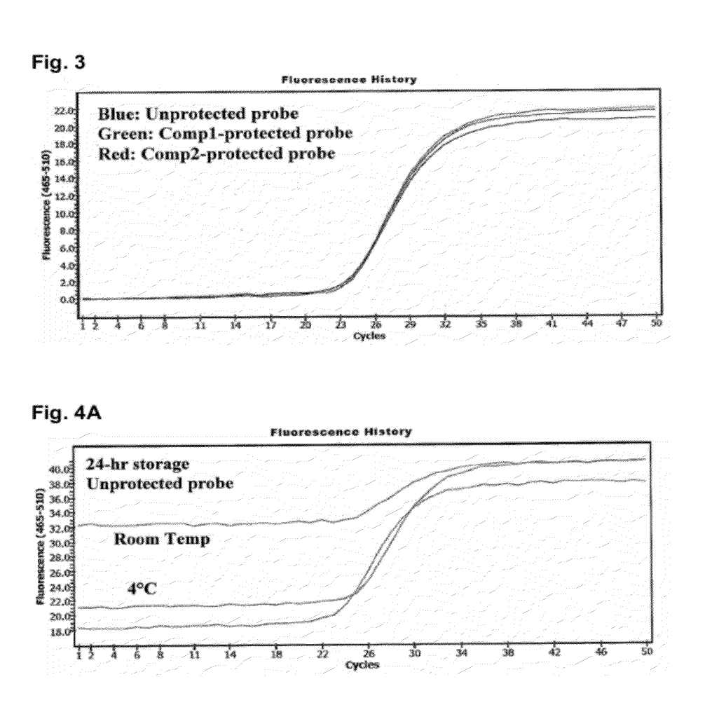

FIG. 3 is a graph showing results of real-time PCR performed with a reaction mixture containing both a protected probe and an unprotected probe. Referring to FIG. 3, it can be seen that the protected probe did not interfere with amplification of a target sequence by PCR or detection of the target sequence by cleaving a nucleic acid probe by RNase H. Such results show that a nucleic acid probe, a complementary strand, and two primers did not interfere with each other during amplification.

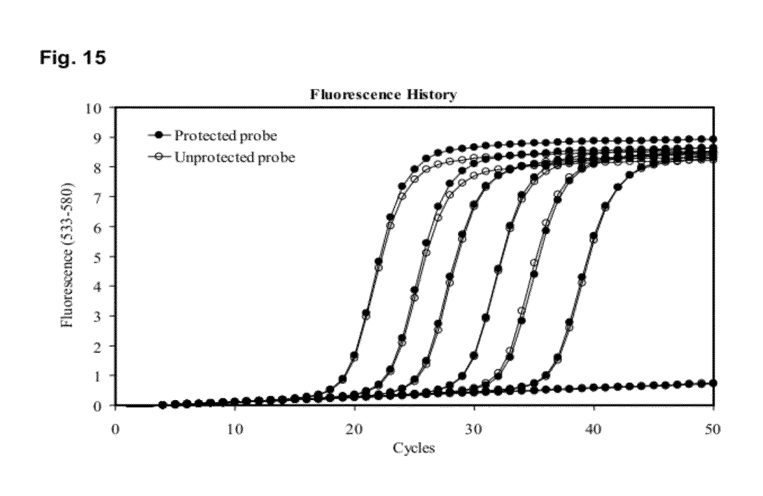

The same reaction conditions were then used to detect E. coli 0157:H7 target DNA sequences by real time PCR. The concentration of E. coli plasmid DNA ranged from 10 copies/reaction to 10⁶ copies/reaction. The primers, probe and probe protector are described in Example 2 below. Results are shown in FIG. 15. FIG. 15 suggests that incorporation of a probe protector doesn't affect the sensitivity of the original assay in which the probe is not protected at least not for the selected target nucleic acid sequences and probes tested.

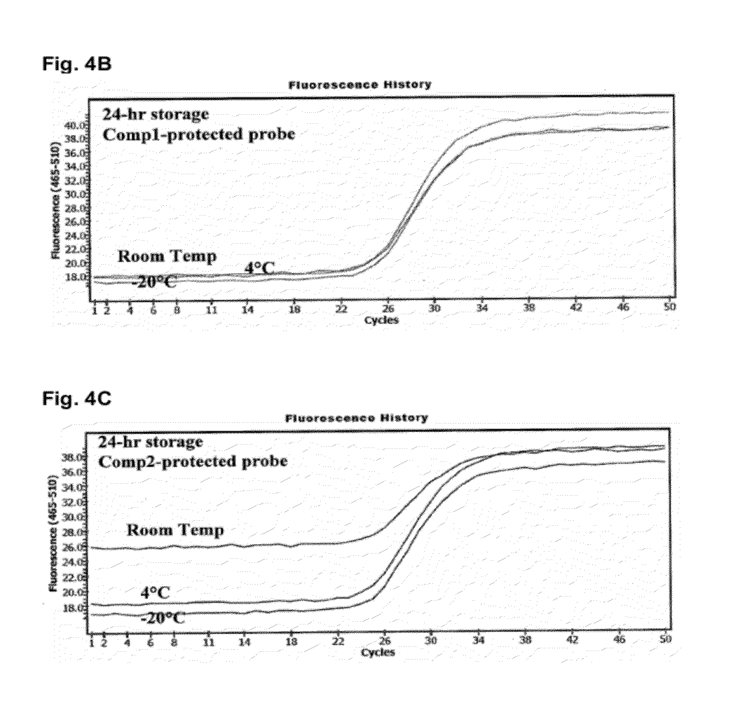

FIGS. 4 A-C show real-time PCR results after an unprotected probe (FIG. 4A) and a probe protected with the RNA-comp1 (FIG. 4B) or the RNA-comp2 (FIG. 4C) were stored at different temperatures for 24 hours. All the components of a real-time PCR mixture except for the template DNA were stored in a master mix form. In general, a higher starting background fluorescence (for example, the average baseline fluorescence intensity over cycles 1-22 at room temperature versus −20° C. in FIG. 4C) indicates that some CATACLEAVE™ probe has become degraded due to RNA hydrolysis. Referring to FIGS. 4 A-C, stability of the protected probe was maintained for 24 hours and the protected probe did not interfere with PCR.

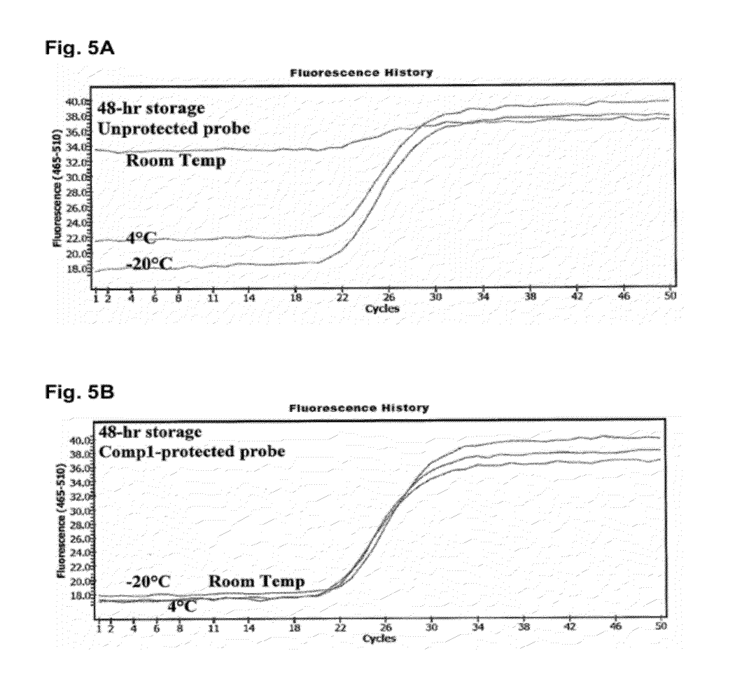

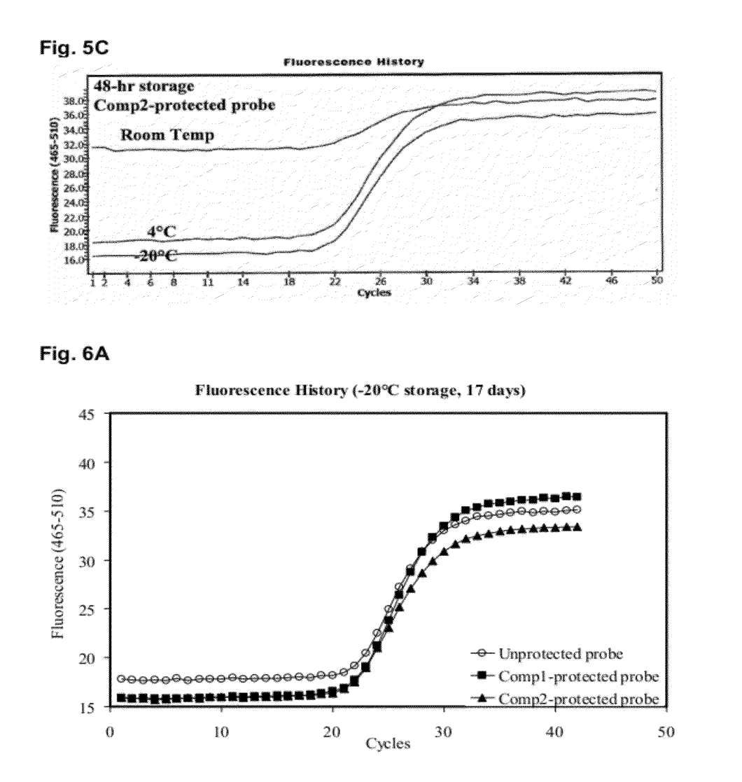

FIGS. 5 A-C show real-time PCR results after an unprotected probe (FIG. 5A) and a probe protected with the RNA-comp1 (FIG. 5B) or the RNA-comp2 (FIG. 5C) were stored at different temperatures for 48 hours. All the components of a real-time PCR mixture except for the template DNA were stored in a master mix form. Referring to FIGS. 5-7, stability of the protected probe was maintained for 48 hours and the protected probe did not interfere with PCR.

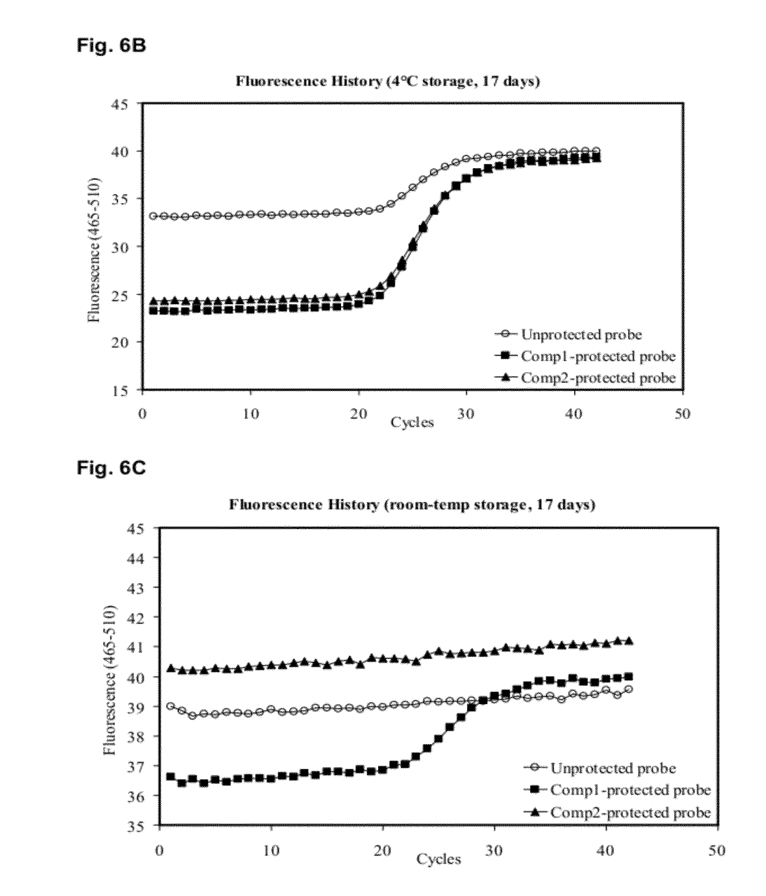

FIGS. 6 A-C show real-time PCR results after unprotected Listeria monocytogenes probes and Listeria monocytogenes probes protected with the RNA-comp1 or the RNA-comp2 were stored at −20° C. (FIG. 6A), 4° C. (FIG. 6B) and room temperature (FIG. 6C) for 17 days. All the components of a real-time PCR mixture except for the template DNA were stored in a master mix form. Referring to FIGS. 6 A-C, the protected probe was relatively stable at −20° C. (FIG. 6A) and 4° C. (FIG. 6B). However, at room temperature (FIG. 6C), the unprotected probe and probe protected with RNA-comp2 became inactive. The probe protected with RNA-comp1 did retain some cleavage activity, but the baseline fluorescence (cycles 1-20) was much higher than that seen in the unprotected probe at −20° C.

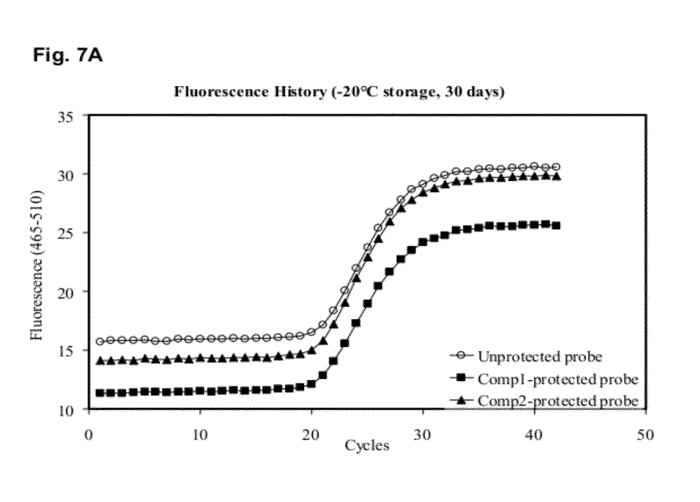

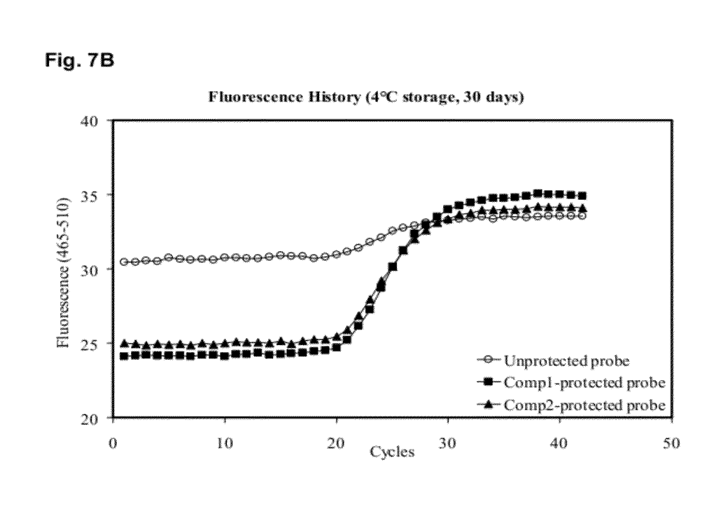

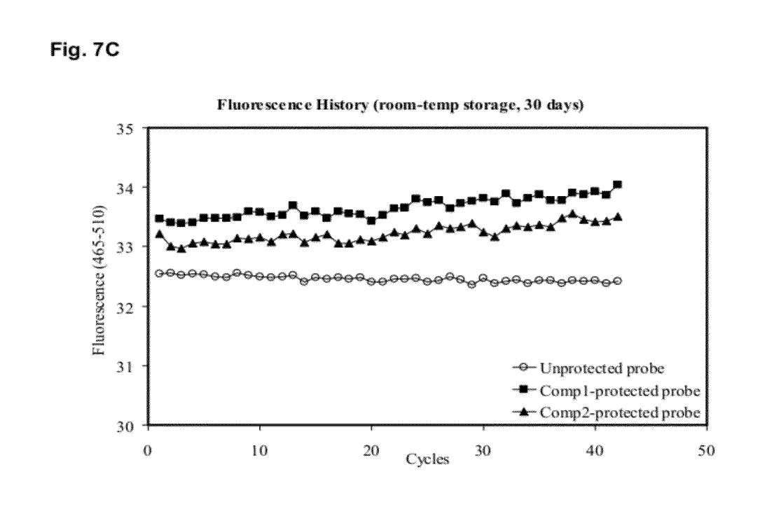

FIGS. 7 A-C show real-time results after an unprotected probe and a probe protected with the RNA-comp1 or RNA-comp2 were stored at −20° C. (FIG. 7A), 4° C. (FIG. 7B), and room temperature (FIG. 7C) for 30 days. All the components of a real-time PCR mixture except for the template DNA were stored in a master mix form. Referring to FIGS. 7 A-C, the protected probe was relatively stable at the temperatures of −20° C. (FIG. 7A) and 4° C. (FIG. 7B). After thirty days at room temperature all three probes became inactive (FIG. 7C).

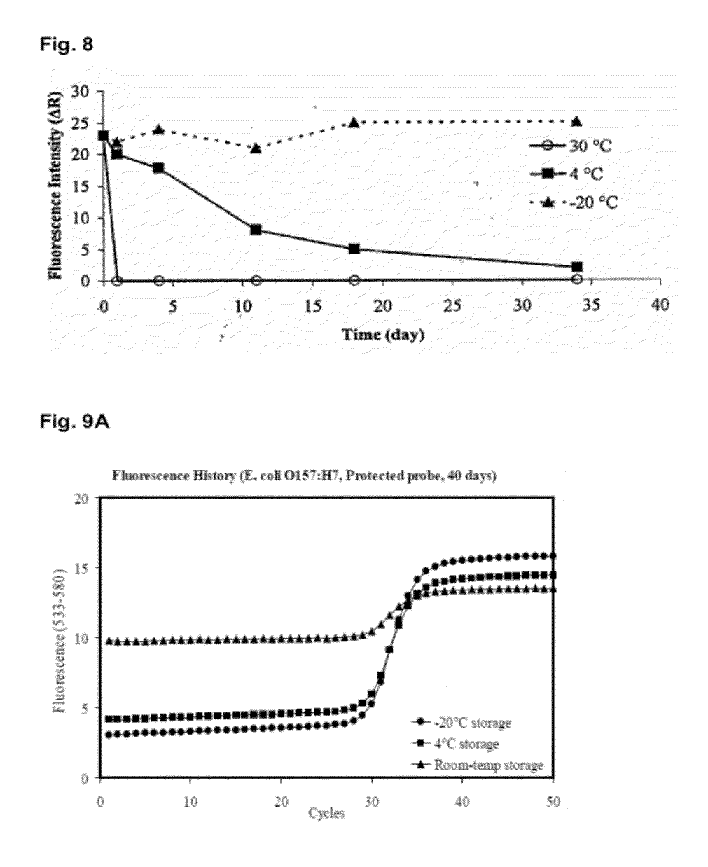

FIG. 8 shows the relative intensity change of fluorescent signals of real-time PCR results after an unprotected probe was stored under different conditions. All the components of a real-time PCR mixture except for the template DNA were stored in a master mix form. Referring to FIG. 8, a sample that had been stored at a temperature of −20° C. was subjected to 6 cycles of freezing and thawing before being used in this experiment. In FIG. 8, ΔR represents normalized fluorescence intensity. Referring to FIG. 8, the unprotected probe that had been stored at a temperature of −20° C. was stable even after the 6 cycles of freezing and thawing. However, at elevated temperatures, the unprotected probe became progressively less stable due to hydrolysis.

### Example 2

### Identification of Protection Effect According to Probe Sequence

In this experiment, nucleic acid probes specific for E. coli, Salmonella, and Listeria spp. were protected with corresponding complementary strand and then stored and the storage effects were identified. The primers, probes, and complementary strand used are as follows:

(SEQ ID NO: 7)
E.coli O157: H7 forward primer: AACGAGCTGTATGTCGTGAGAATC
(SEQ ID NO: 8)
E.coli O157: H7 reverse primer: ATGGATCATCAAGCTCTAAGAAAGAAC
(SEQ ID NO: 9)
E.coli O157: H7 probe: ATAGGCTTrGrArArGCAGTGCAT
(SEQ ID NO: 10)
E.coli O157: H7 complementary strand: rCrU rGrCrU rUrCrA rArGrC
(SEQ ID NO: 11)
Salmonella forward primer: TCG TCA TTC CAT TAC CTA CC
(SEQ ID NO: 12)
Salmonella reverse primer: TAC TGA TCG ATA ATG CCA GAC GAA
(SEQ ID NO: 13)
Salmonella probe: CGA TCA GrGrA rArAT CAA CCA G
(SEQ ID NO: 14)
Salmonella complementary strand: rGrGrU rUrGrA rUrUrU rCrCrU rGrArU
(SEQ ID NO: 15)
Listeria forward primer: TCCAAGCAGTGAGTGTGAGAA
(SEQ ID NO: 16)
Listeria reverse primer: TGACAGCGTGAAATCAGGA
(SEQ ID NO: 17)
Listeria probe: CCATCACAGCTCArUGCTTCGC
(SEQ ID NO: 18)
Listeria complementary strand: rGrArA rGrCrA rUrGrA rGrC

The compositions of the PCR mixture and PCR reaction conditions were the same as in Example 1, except that the primers, probes, and complementary strand had different sequences and different template DNA was used. In regard to the template DNA, plasmid DNA including an E. coli target sequence, plasmid DNA including a Salmonella target sequence, and plasmid DNA including a Listeria target sequence were used.

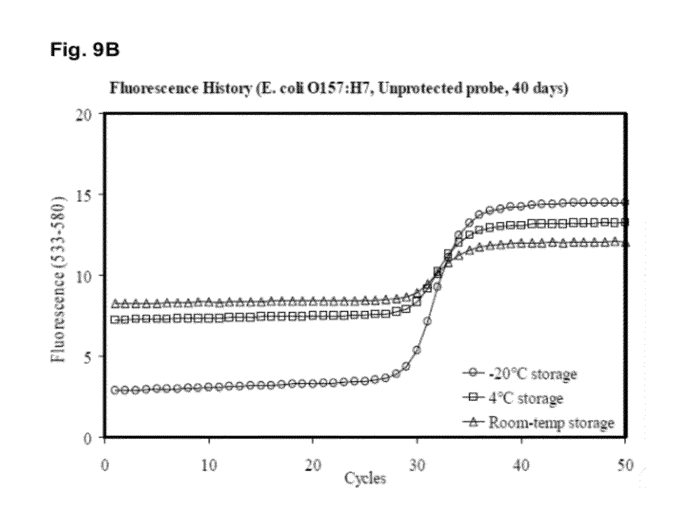

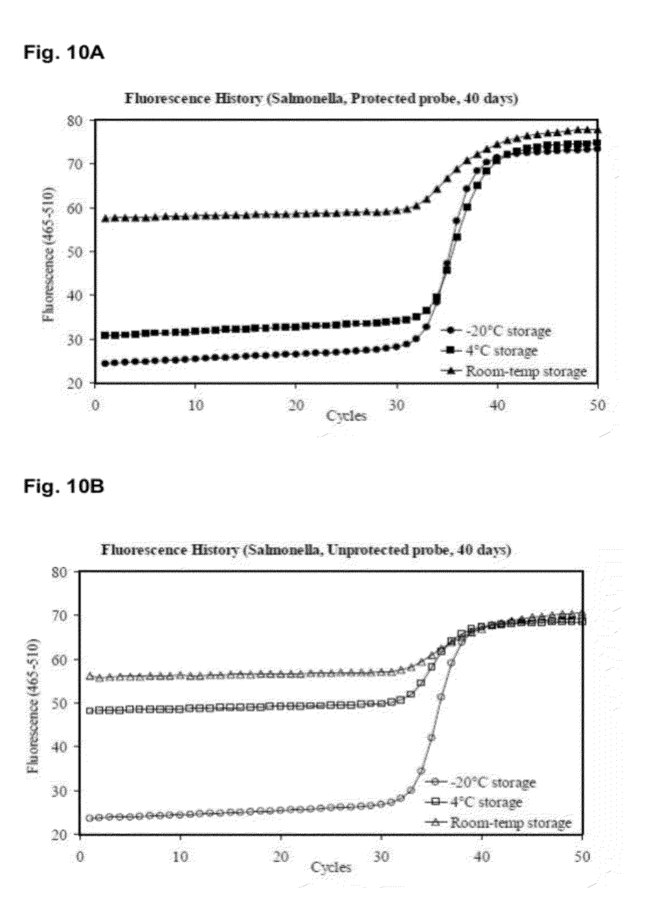

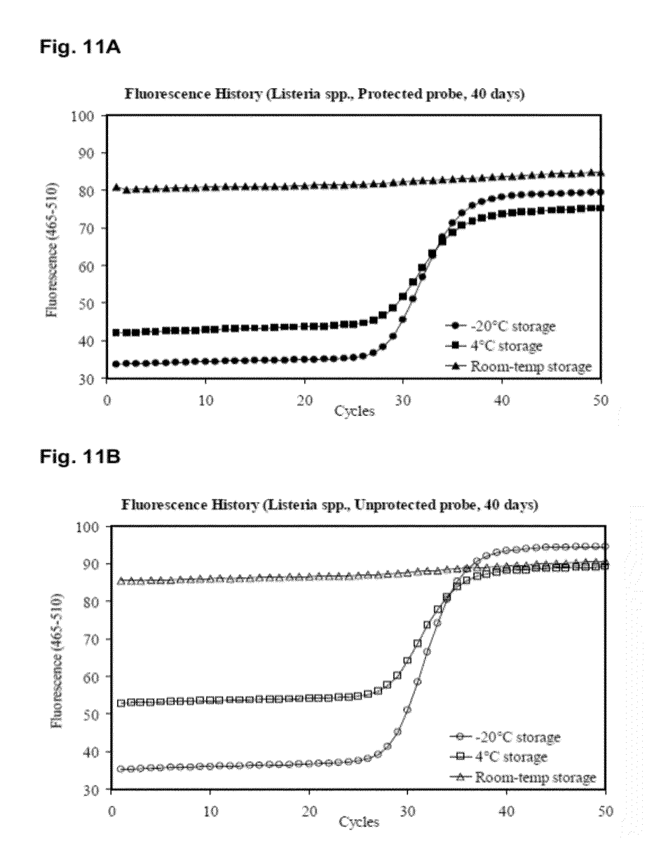

FIGS. 9A to 11B show real-time PCR results after using E. coli-specific, Salmonella-specific, and Listeria-specific unprotected probes and probes protected with a complementary strand were stored at −20° C., 4° C., and 30° C. for 40 days. All the components of a real-time PCR mixture except for the template DNA were stored in a master mix form. Referring to FIGS. 10 and 11, the protected probe was relatively stable at −20° C., 4° C., and room temperature. In all cases the protected probe showed significantly slower degradation kinetics than the unprotected one. Such results show that even when a probe sequence is changed, stability of the probe is maintained for a relatively long period of time over a wide temperature range.

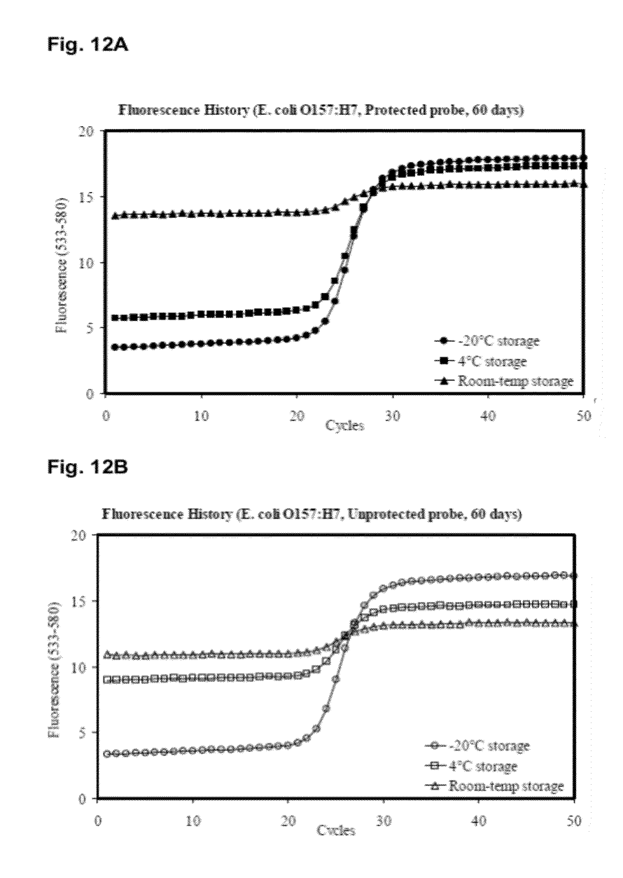

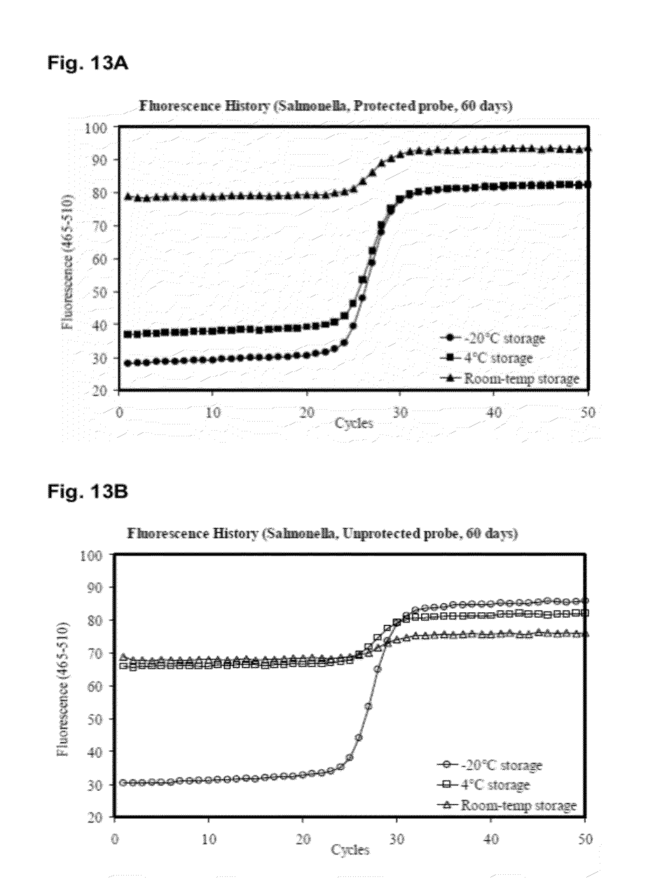

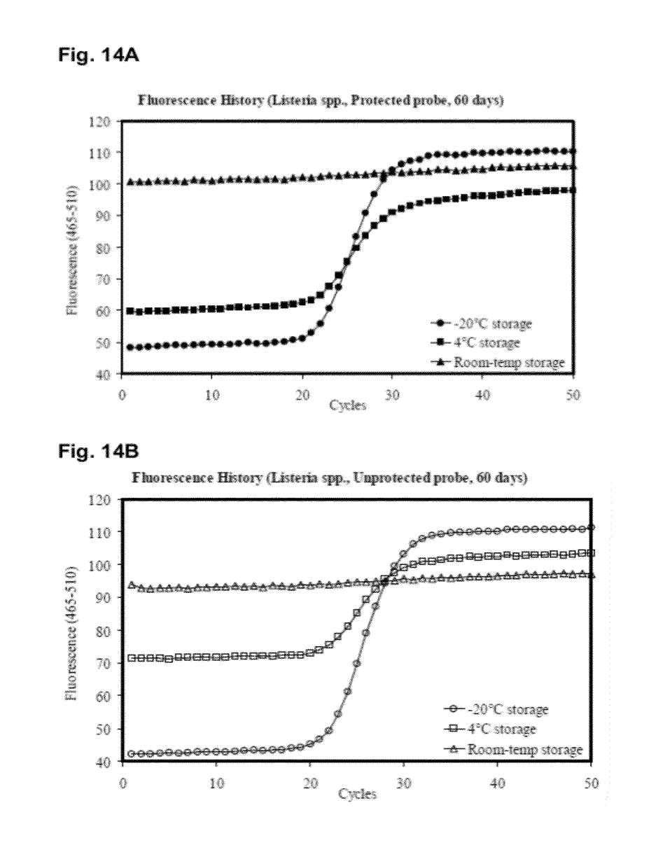

FIGS. 12A to 14B show real-time PCR results after using E. coli-specific, Salmonella-specific, and Listeria-specific unprotected probe and a probe protected with a complementary strand were stored at −20° C., 4° C., and 30° C. for 60 days. All the components of a real-time PCR mixture except for the template DNA were stored in a master mix form. Referring to FIGS. 12 to 14, the protected probe was relatively stable at −20° C., 4° C., and at room temperature. In all cases the protected probe showed significantly slower degradation kinetics than the unprotected one. Such results show that even when a probe sequence is changed, stability of the probe is maintained for a relatively long period of time.

1. A probe protector oligonucleotide having the structure of R3-X′—R4 (Formula II) that is substantially complementary to an oligonucleotide probe having the structure of R1-X—R2 (Formula I), wherein each of R1, R2, R3, and R4 is selected from a nucleic acid or a nucleic acid analog and each of X and X′ is a naturally occurring or modified RNA, and wherein said oligonucleotide probe nucleic acid sequence is substantially complementary to a target nucleic acid sequence.

2. The probe protector oligonucleotide of claim 1, wherein a melting temperature, Tm, of the probe protector oligonucleotide hybridizing with the oligonucleotide probe is lower than a melting temperature, Tm′, of said oligonucleotide probe hybridizing with the target nucleic acid sequence.

3. The probe protector oligonucleotide of claim 2, wherein the melting temperature Tm of the probe protector oligonucleotide hybridizing to the oligonucleotide probe is lower by about 10° C. as compared to the melting temperature Tm′ of the oligonucleotide probe hybridizing to said target nucleic acid sequence.

4. A protected probe comprising the oligonucleotide probe of claim 1 base paired with the probe protector oligonucleotide of claim 1.

5. A method of stabilizing an oligonucleotide probe comprising the steps of hybridizing the probe protector probe of claim 1 with the oligonucleotide probe of claim 1, wherein said hybridization stabilizes said oligonucleotide probe.

6. A method for the real-time detection of a target DNA sequence in a sample, comprising the steps of:
providing a sample comprising a target DNA sequence;
providing a pair of forward and reverse amplification primers that can anneal to the target DNA sequence;
providing a protected probe comprising a labeled oligonucleotide probe base-paired to a protected probe oligonucleotide, wherein said oligonucleotide probe comprises RNA and DNA nucleic acid sequences that are substantially complementary to the target DNA sequence;
amplifying a PCR fragment between the forward and reverse amplification primers in the presence of an amplifying polymerase activity, an amplification buffer, and an RNase H activity and the protected probe under conditions where said oligonucleotide probe can dissociate from said protector probe oligonucleotide and the RNA sequences of the oligonucleotide probe can form a RNA:DNA heteroduplex with the target DNA sequence present in the PCR fragment, and
detecting a real-time increase in the emission of a signal from the label on the oligonucleotide probe,
wherein the increase in signal indicates the presence of the target DNA sequence in the sample.

7. The method of claim 6, wherein said protected probe comprises:
a labeled oligonucleotide probe having the structure of R1-X—R2 (Formula I) base-paired with a probe protector oligonucleotide having the structure of R3-X′—R4 (Formula II), wherein each of R1, R2, R3, and R4 is selected from a nucleic acid or a nucleic acid analog, each of X and X′ is a naturally occurring or modified RNA, and wherein said probe protector oligonucleotide is substantially complementary to said oligonucleotide probe.

8. The method of claim 7, wherein a melting temperature, Tm, of the probe protector oligonucleotide hybridizing with the oligonucleotide probe is lower than a melting temperature, Tm′, of said oligonucleotide probe hybridizing with the target nucleic acid sequence.

9. The method of claim 8, wherein the melting temperature Tm of the probe protector oligonucleotide hybridizing to the oligonucleotide probe is lower by about 10° C. as compared to the melting temperature Tm′ of the oligonucleotide probe hybridizing to said target nucleic acid sequence.

10. The method of claim 6, wherein the real-time increase in the emission of the signal from the label on the oligonucleotide probe results from the RNase H cleavage of the heteroduplex formed between the oligonucleotide probe and one of the strands of the PCR fragment.

11. The method of claim 6, wherein said amplifying of the PCR fragment occurs under conditions where said oligonucleotide probe can dissociate from said protector probe oligonucleotide and the RNA sequences of the oligonucleotide probe can form a RNA: DNA heteroduplex with the target DNA sequence present in the PCR fragment.

12. The method of claim 6, wherein the oligonucleotide probe is labeled with a fluorescence resonance energy transfer (FRET) pair comprising a fluorescence donor and a fluorescence acceptor.

13. A kit for the real-time detection of a target nucleic acid sequences in a sample comprising
a probe protector oligonucleotide having the structure of R3-X′—R4 (Formula II) and oligonucleotide probe having the structure of R1-X—R2 (Formula I),
wherein the probe protector oligonucleotide is substantially complementary to an oligonucleotide probe, each of R1, R2, R3, and R4 is selected from a nucleic acid or a nucleic acid analog, each of X and X′ is a naturally occurring or modified RNA, and
wherein said oligonucleotide probe nucleic acid sequence is substantially complementary to a target nucleic acid sequence.

14. The kit of claim 13, wherein a melting temperature (Tm) of the probe protector oligonucleotide hybridizing with the oligonucleotide probe is lower than a melting temperature Tm′ of said oligonucleotide probe hybridizing with the target nucleic acid sequence.

15. The kit of claim 14, wherein the melting temperature Tm of the probe protector oligonucleotide hybridizing to the oligonucleotide probe is lower by about 10° C. as compared to the melting temperature Tm′ of the oligonucleotide probe hybridizing to said target nucleic acid sequence.

16. The kit of claim 13, further comprising positive internal and negative controls.

17. The kit of claim 13, wherein the probe is labeled with a FRET pair.

18. The kit of claim 13, wherein the kit further comprises an amplifying polymerase activity.

19. The kit of claim 13, wherein the kit further comprises a reverse transcriptase activity for the reverse transcription of a target RNA sequence to produce a target cDNA sequence.

20. The kit of claim 13, wherein the kit further comprises an RNase H activity.
